(19) United States
(12) Patent Application Publication
(10) Pub. No.: US 2010/0280433 A1
(43) Pub. Date: Nov. 4, 2010

(54) PROSTATE GLAND ELECTROCHEMICAL-THERAPEUTIC DEVICE

(76) Inventors: Wan Hung Lai, Taipei City (TW); Cheng Zung Liao, Taipei County (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 12/718,566

(22) Filed: Mar. 5, 2010

(30) Foreign Application Priority Data
May 4, 2009 (TW) 098207538

(51) Int. Cl. A61N 1/30

(52) U.S. Cl. 604/21

(57) ABSTRACT

A prostate gland electrochemical-therapeutic device is provided. The prostate gland electrochemical-therapeutic device includes a probe unit and an electrode unit. The probe unit is adapted for introducing a saline solution into a patient's prostate gland. The electrode unit then conducts an electrochemical reaction on the saline solution introduced in the prostate gland to generate a strong acid solution and/or a strong alkaline solution for curing the prostate gland thereby.

### BACKGROUND OF THE INVENTION

1. Field of the Invention

The present invention relates generally to a therapeutic device for curing prostatomegaly, and more particularly, to a prostate gland electrochemical-therapeutic device adapted for curing prostatomegaly symptom.

2. The Prior Arts

It is well known that when a direct current is applied between a positive electrode and a negative electrode positioned in a saline solution, the saline solution adjacent to the positive electrode presents a strong acidity, and the saline solution adjacent to the negative electrode presents a strong alkalinity. The saline solution presenting strong acidity obtained in this manner has outstanding performance in many applications. For example, it can be employed in disinfectant fluid, astringent cleaner, or for substituting metal cutting oil. It may even be used for agriculture purpose. For example, it can be absorbed by roots of plants. Therefore, the saline solution presenting strong acidity can be used as growth improving agent, disinsecting agent, for improving the harvest of crops, or even improving the taste or sugar degree of the crops. Furthermore, the saline solution presenting strong acidity or strong alkalinity has been applied for medical treatment and therapy, such as preventing MRSA infection, curing dermatitis, and cleaning and sterilizing endoscope.

Generally, many organ removal operations which were typical surgical operations can be now conducted as percutaneous operations or laparoscopic operations in which the patients suffer much less physical and/or mental pains.

Prostatomegaly symptom is often cured via the urethra, in that an electrotome, a laser probe or a freezing probe is inserted through the urethra to the prostate gland to conduct a resection operation or providing liquid nitrogen thereby for freezing treatment.

Recently, microwave is also used as a therapeutic approach in curing prostatomegaly symptom. Specifically, a probe having several microwave antennas positioned therearound is inserted into the urethra, and microwaves are emitted from the antennas for heating treatment. Or alternatively, a part of the probe is provided with a conductor, so as to concentrate the generated heat, thus providing a more efficient heating treatment thereby.

However, there are several therapeutic methods, such as the transurethral warm temperature treatment which heating temperature range is 41° C. to 44° C., the transurethral high temperature treatment which heating temperature range is higher than 50° C., and the transurethral ablation method which heating temperature is higher than 70° C. to vaporize the enlargement portion of the prostate gland. Further, the transurethral warm temperature treatment and transurethral high temperature treatment are also proposed to be used in curing rectum diseases.

Unfortunately, all of the aforementioned therapeutic methods for curing the prostatomegaly symptom would not only more or less impair the urethra, but also extremely irritate the patient so that a local even a general anesthesia is required. However, the patients of the prostatomegaly symptom are often seniors who also suffer other diseases such as hypertension, heart disease, or diabetes. When such senior patients are conducted with anesthesia, severe side effect may also occur, and therefore many patients are not encouraged or dare not to take the foregoing treatments.

### SUMMARY OF THE INVENTION

A primary objective of the present invention is to provide a prostate gland electrochemical-therapeutic device. When a patient is cured with such a prostate gland electrochemical-therapeutic device, the anesthesia is not required, the patient suffers much less pain, and the urethra can be protected from being impaired.

For achieving the foregoing objective and others, the present invention provides a prostate gland electrochemical-therapeutic device. The prostate gland electrochemical-therapeutic device includes a probe unit and an electrode unit. The probe unit includes a front portion, a rear portion, a urine flow duct, and a balloon unit. The front portion is configured with a urination orifice, and the rear portion is configured with a solution inlet. The urine flow duct has a urine flow orifice configured at the front portion, and a urine drawing orifice configured at the rear portion and at an opposite side of the urine flow duct relative to the urine flow orifice. The balloon unit includes a balloon solution inlet configured at the rear portion, a balloon configured at the front portion, and a plurality of solution supply orifices configured at the front portion adjacent to the urination orifice. The balloon unit controls the balloon to inflate or deflate by inputting or outputting a distilled water from the balloon solution inlet. The balloon is made by a flexible material.

The electrode unit has a switchable circuit structure and includes a power supply, a plurality of positive electrodes, a plurality of negative electrodes, a plurality of electrode elements, and a plurality of pH value sensors. The power supply is electrically connected with the positive electrodes and the negative electrodes. The positive electrodes and the negative electrodes are electrically coupled to the electrode elements via the rear portion. The electrode members are made of a shape memory alloy material, and include a layer of perfect conductive material plated thereon. The shapes of the electrode members can be arbitrarily varied so as for being compatible for any patient. The pH value sensors are provided together with the electrode elements at the solution supply orifices. The pH value sensors are adapted for instantly obtaining the pH values during the treatment or after the treatment. The detected pH values can be accorded for adjusting the current intensity, or the practical pH values. Each negative electrode is coated with an ion exchange membrane. The ion exchange membrane is adapted to insulate the positive electrode from the negative electrode, and allows anions and cations exchanging thereby, so as to further improve the strong acidity and the strong alkalinity.

In operation, the front portion of the probe unit is inserted through the urethra into the prostate gland and the urinary bladder, until the solution supply orifices reach the prostate gland and the balloon reaches the urinary bladder. Then, a distilled water is inputted from the balloon solution inlet into the balloon so as to inflate the balloon. Then, a saline solution is filled therein from the solution inlet, during which the saline solution is guided from the rear portion to the front portion and out from the solution supply orifices and rest in the prostate gland. Then, the power supply is turned on, and the switchable circuit is adjusted, so as to maintain the positive electrodes and the negative electrodes conducted with a direct current to the electrode elements. An electrochemical reaction is raised by the electrode elements to the saline solution rested in the prostate gland, thus generating solutions with strong acidity and strong alkalinity respectively. The solutions with strong acidity and strong alkalinity respectively rest nearby the positive electrodes and the negative electrodes, respectively. They function to treat or cure the prostate gland thereby.

### BRIEF DESCRIPTION OF THE DRAWINGS

The present invention will be apparent to those skilled in the art by reading the following detailed description of preferred embodiments thereof, with reference to the attached drawings, in which:

FIG. 1A is a schematic diagram illustrating a probe unit of a prostate gland electrochemical-therapeutic device according to an embodiment of the present invention;

FIG. 1B is a partial enlargement view of the prostate gland electrochemical-therapeutic device according to an embodiment of the present invention; and

FIG. 2 is a schematic diagram illustrating an operation status of the prostate gland electrochemical-therapeutic device according to an embodiment of the present invention.

### DETAILED DESCRIPTION OF THE PREFERRED EMBODIMENT

The accompanying drawings are included to provide a further understanding of the invention, and are incorporated in and constitute a part of this specification. The drawing illustrates embodiments of the invention and, together with the description, serves to explain the principles of the invention.

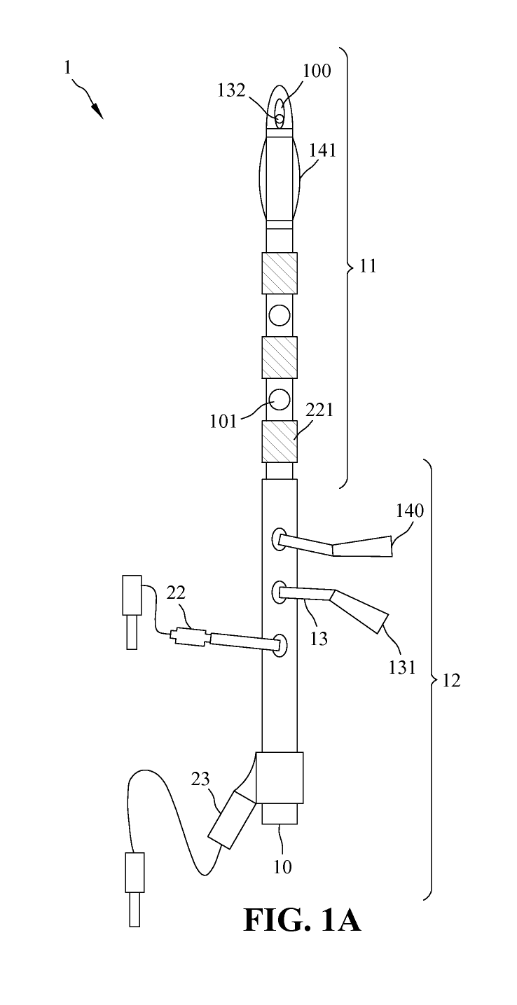

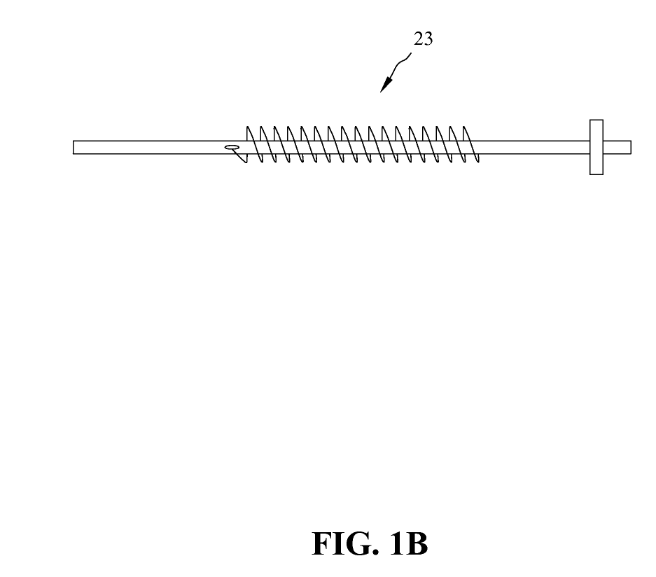

FIG. 1A is a schematic diagram illustrating a probe unit of a prostate gland electrochemical-therapeutic device according to an embodiment of the present invention. FIG. 1B is a partial enlargement view of the prostate gland electrochemical-therapeutic device. Referring to FIGS. 1A and 1B, the prostate gland electrochemical-therapeutic device includes a probe unit 1, and an electrode unit 2.

The probe unit 1 includes a front portion 11, a rear portion 12, a urine flow duct 13, and a balloon unit 14. The front portion 11 is configured with a urination orifice 100, and the rear portion is configured with a solution inlet 10. The urine flow duct 13 has a urine flow orifice 132 configured at the front portion 11, and a urine drawing orifice 131 configured at the rear portion 12 and at an opposite side of the urine flow duct 13 relative to the urine flow orifice 132. The balloon unit 14 includes a balloon solution inlet 140 configured at the rear portion 12, a balloon 141 configured at the front portion 11, and a plurality of solution supply orifices 101 configured at the front portion 11 adjacent to the urination orifice 100. The balloon unit 14 controls the balloon 141 to inflate or deflate by inputting or outputting a distilled water from the balloon solution inlet 140. The balloon is made by a flexible material.

The electrode unit 2 has a switchable circuit structure (not shown in the drawings) and further includes a power supply 21, a plurality of positive electrodes 22, a plurality of negative electrodes 23, a plurality of electrode elements 221, and a plurality of pH value sensors (not shown in the drawings). The switchable circuit is adapted to switch or control the positive electrodes 22 or negative electrodes 23. The power supply 21 is electrically connected with the positive electrodes 22 and the negative electrodes 23. The positive electrodes 22 and the negative electrodes 23 are electrically coupled to the electrode elements 221 via the rear portion 12. The electrode members 221 are made of a shape memory alloy material, and include a layer of perfect conductive material, e.g., metal, plated thereon. The shapes of the electrode members can be arbitrarily varied so as for being compatible for any patient. The pH value sensors are provided together with the electrode elements 221 at the solution supply orifices 101. The pH value sensors are adapted for instantly obtaining the pH values during the treatment or after the treatment. The detected pH values can be accorded for adjusting the current intensity, or the practical pH values. Each negative electrode 23 is coated with an ion exchange membrane (not shown in the drawings). The ion exchange membrane is adapted for insulating the positive electrode 22 from the negative electrode 23, and allows anions and cations exchanging thereby, so as to further improve the strong acidity and the strong alkalinity.

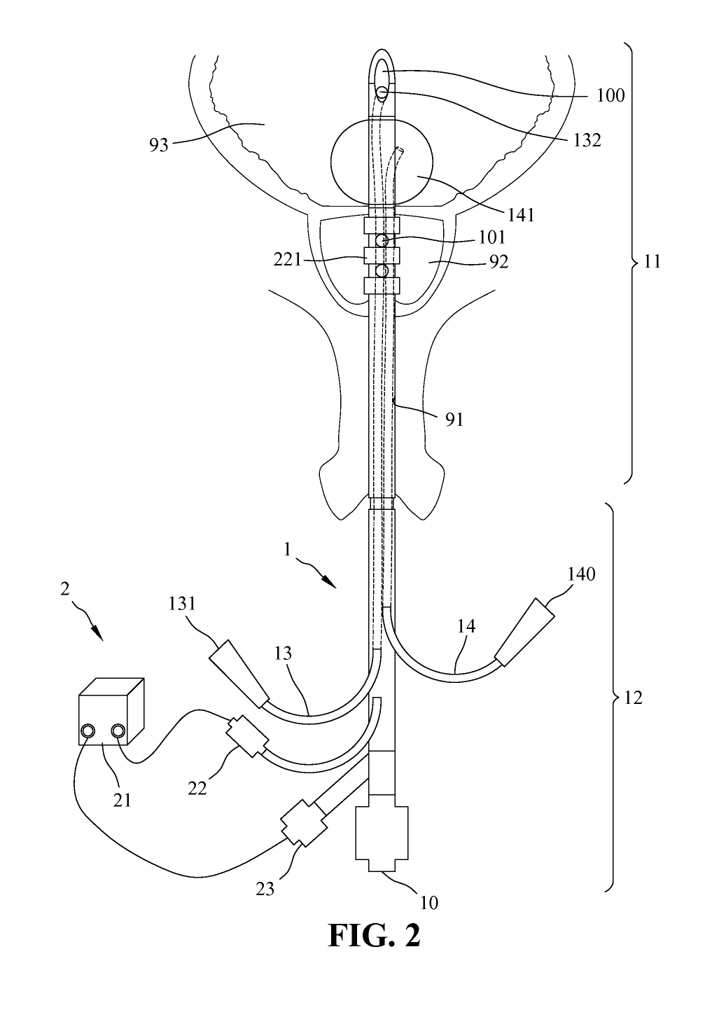

FIG. 2 is a schematic diagram illustrating an operation status of the prostate gland electrochemical-therapeutic device according to an embodiment of the present invention. Referring to FIG. 2, in operation, the front portion 11 of the probe unit 1 is inserted through patient's urethra 91 into the prostate gland 92 and the urinary bladder 93, until the solution supply orifices 101 reach the prostate gland 92 and the balloon 141 reaches the urinary bladder 93. Then, a distilled water is inputted from the balloon solution inlet 140 into the balloon 141 so as to inflate the balloon 141. Then, a saline solution is filled therein from the solution inlet 10, during which the saline solution is guided from the rear portion 12 to the front portion 11 and out from the solution supply orifices 101 and rest in the prostate gland 92. Then, the power supply 21 is turned on, and the switchable circuit is adjusted, so as to maintain the positive electrodes 22 and the negative electrodes 23 conducted with a direct current to the electrode elements 221. An electrochemical reaction is raised by the electrode elements 221 to the saline solution rested in the prostate gland 92, thus generating solutions with strong acidity and strong alkalinity respectively. The solutions with strong acidity and strong alkalinity respectively rest nearby the positive electrodes 22 and the negative electrodes 23, respectively. They function to treat or cure the prostate gland 92 thereby.

Preferably, the treatment lasts for 1.5 to 2 hours, and the prostate gland tissue can be reduced to about 5 millimeters to several centimeters. When a patient is cured with such a prostate gland electrochemical-therapeutic device, the anesthesia is not required, the patient suffers much less pain, and the urethra can be protected from being impaired.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A prostate gland electrochemical-therapeutic device, comprising:
a probe unit, comprising:
a front portion, configured with a urination orifice;
a rear portion, configured with a solution inlet;
a urine flow duct, having a urine flow orifice configured at the front portion, and a urine drawing orifice configured at the rear portion and at an opposite side of the urine flow duct relative to the urine flow orifice; and
a balloon unit, comprising a balloon solution inlet configured at the rear portion, a balloon configured at the front portion, and a plurality of solution supply orifices configured at the front portion adjacent to the urination orifice; and
an electrode unit, comprising:
a plurality of positive electrodes;
a plurality of negative electrodes; and
a power supply, electrically connected to the positive electrodes and the negative electrodes.

2. The prostate gland electrochemical-therapeutic device as claimed in claim 1, wherein the electrode unit further comprises a plurality of pH value sensors.

3. The prostate gland electrochemical-therapeutic device as claimed in claim 1, wherein the positive electrodes and the negative electrodes are made of shape memory alloy, and are coated with perfect conductor material thereon.

4. The prostate gland electrochemical-therapeutic device as claimed in claim 1, wherein the positive electrodes and the negative electrodes are coupled with a switch circuit adapted for switching or controlling the positive electrodes or the negative electrodes.

5. The prostate gland electrochemical-therapeutic device as claimed in claim 1, wherein each negative electrode is provided with an ion exchange membrane.
